(19) United States
(12) Patent Application Publication
(10) Pub. No.: US 2018/0317970 A1
(43) Pub. Date: Nov. 8, 2018

(54) SPINAL RODS WITH MARKINGS, AND RELATED SYSTEMS AND METHODS

(71) Applicant: Medos International Sarl, Le Locle (CH)

(72) Inventors: Roman Lomeli, Plymouth, MA (US); Kevin Lee, Canton, MA (US)

(21) Appl. No.: 15/588,265

(22) Filed: May 5, 2017

(51) Int. Cl. A61B 17/70; A61B 90/00; B21F 45/00

(52) U.S. Cl. CPC A61B 17/7031; A61B 90/39; A61B 2017/00526; A61B 2090/3937; B21F 45/008

(57) ABSTRACT

A method of preparing a spinal rod includes obtaining a body that is elongate along a central rod axis and includes a proximal end and a distal end spaced from each other along a longitudinal direction to as to define the spinal rod. The body defines an outer surface extending between the proximal and distal ends and the body comprises a material that is malleable so as to allow the spinal rod to be bent to a predetermined curvature. The method includes producing at least one longitudinal line on the outer surface, such that the at least one longitudinal line is elongate along the longitudinal direction and parallel with the central rod axis. The method also includes producing a plurality of harsh marks incrementally spaced along the outer surface and producing reference numbers on the outer surface. Each of the reference numbers identifies a distance along the longitudinal direction from the proximal end of the body to a respective one of the hash marks.

### FIELD OF THE DISCLOSURE

The present disclosure generally relates to spinal rods, and in particular relates to spinal rods that have reference markings thereon.

### BACKGROUND

Bone anchors and spinal rods and methods of their use in treating spinal disorders are known. Typical methods involve anchoring at least two screws into the vertebrae, and fixing the screws along a spinal rod to position or immobilize the vertebrae with respect to one another. The screws commonly have anchor heads with U-shaped channels in which the spinal rod is inserted and subsequently clamped by a fastener, such as, for example, a threaded nut, set screw or locking cap. These methods commonly involve multiple screws and multiple spinal rods. The spinal rods can be shaped to maintain the vertebrae in a desired orientation so as to correct the spinal disorder at hand (e.g., to straighten a spine having abnormal curvature). Additionally or alternatively, the screws may be spaced along the rods(s) to compress or distract adjacent vertebrae. To successfully correct the curvature of a spine, the spinal rods can be bent by the physician into the desired shape, which bending can occur during the implantation procedure.

### SUMMARY

In accordance with one embodiment, a method of preparing a spinal rod includes obtaining a body that is elongate along a central rod axis and includes a proximal end and a distal end spaced from each other along a longitudinal direction to as to define the spinal rod. The body defines an outer surface extending between the proximal and distal ends and the body comprises a material that is malleable so as to allow the spinal rod to be bent to a predetermined curvature. The method includes producing at least one longitudinal line on the outer surface, such that the at least one longitudinal line is elongate along the longitudinal direction and parallel with the central rod axis. The method also includes producing a plurality of harsh marks incrementally spaced along the outer surface and producing reference numbers on the outer surface. Each of the reference numbers identifies a distance along the longitudinal direction from the proximal end of the body to a respective one of the hash marks.

In accordance with a further embodiment, a method of preparing a spinal rod for implantation includes obtaining a template rod and an implantation rod each defining a central axis and having an outer surface that defines: 1) at least one line elongated along a longitudinal direction that is parallel with the central axis; 2) hash marks spaced at intervals along the longitudinal direction; and 3) reference numbers identifying a distance from a proximal end of the associated rod to a respective one of the hash marks, measured along the longitudinal direction. The method includes bending the template rod so as to form a first region defining a first bent shape and, after bending the template rod, comparing at least one of the hash marks and associated reference numbers of the template rod with at least one of the hash marks and associated reference numbers of the implantation rod so as to locate a second region of the implantation rod for bending. The method also includes bending the implantation rod at the second region so as to form a second bent shape and comparing a curvature of the at least one line of the template rod with a curvature of the at least one line of the implantation rod while bending the implantation rod until the second bent shape is substantially equivalent to the first bent shape.

### DESCRIPTION OF THE DRAWINGS

The foregoing summary, as well as the following detailed description of preferred embodiments of the application, will be better understood when read in conjunction with the appended drawings. For the purposes of illustrating the embodiments of the present application, there is shown in the drawings certain embodiments. It should be understood, however, that the application is not limited to the precise arrangements and instrumentalities shown. In the drawings:

FIG. 1 is a perspective view of a spinal rod in a pre-operative configuration, according to a first example embodiment of the present disclosure;

FIG. 2 is another perspective view of the spinal rod of FIG. 1, facing an opposite end of the spinal rod shown in FIG. 1;

FIG. 3 is a longitudinal top view of the spinal rod shown in FIG. 1;

FIG. 4 is a longitudinal bottom view of the spinal rod shown in FIG. 1;

FIG. 5 is a longitudinal side view of a first side of the spinal rod of FIG. 1;

FIG. 6 is a longitudinal side view of a second side of the spinal rod opposite the first side shown in FIG. 5;

FIG. 7 is a front end view of the spinal rod shown in FIG. 1;

FIG. 8 is a rear end view of the spinal rod shown in FIG. 1;

FIG. 9 is a perspective view of a spinal rod in a pre-operative configuration, according to a second example embodiment of the present disclosure;

FIG. 10 is another perspective view of the spinal rod of FIG. 9, facing an opposite end of the spinal rod shown in FIG. 9;

FIG. 11 is a longitudinal top view of the spinal rod shown in FIG. 9;

FIG. 12 is a longitudinal bottom view of the spinal rod shown in FIG. 9;

FIG. 13 is a longitudinal side view of a first side of the spinal rod of FIG. 9;

FIG. 14 is a longitudinal side view of a second side of the spinal rod opposite the first side shown in FIG. 13;

FIG. 15 is a front end view of the spinal rod shown in FIG. 9;

FIG. 16 is a rear end view of the spinal rod shown in FIG. 9;

FIG. 17 is a perspective view of a spinal rod in a pre-operative configuration, according to a third example embodiment of the present disclosure;

FIG. 18 is another perspective view of the spinal rod of FIG. 17, facing an opposite end of the spinal rod shown in FIG. 17;

FIG. 19 is a longitudinal top view of the spinal rod shown in FIG. 17;

FIG. 20 is a longitudinal bottom view of the spinal rod shown in FIG. 17;

FIG. 21 is a longitudinal side view of a first side of the spinal rod of FIG. 17;

FIG. 22 is a longitudinal side view of a second side of the spinal rod opposite the first side shown in FIG. 21;

FIG. 23 is a front end view of the spinal rod shown in FIG. 17;

FIG. 24 is a rear end view of the spinal rod shown in FIG. 17;

FIG. 25 is a perspective view of a spinal rod in a pre-operative configuration, according to a fourth example embodiment of the present disclosure;

FIG. 26 is another perspective view of the spinal rod of FIG. 25, facing an opposite end of the spinal rod shown in FIG. 25;

FIG. 27 is a longitudinal top view of the spinal rod shown in FIG. 25;

FIG. 28 is a longitudinal bottom view of the spinal rod shown in FIG. 25;

FIG. 29 is a longitudinal side view of a first side of the spinal rod of FIG. 25;

FIG. 30 is a longitudinal side view of a second side of the spinal rod opposite the first side shown in FIG. 29;

FIG. 31 is a front end view of the spinal rod shown in FIG. 25;

FIG. 32 is a rear end view of the spinal rod shown in FIG. 25;

FIG. 33 is a perspective view of a spinal rod in a pre-operative configuration, according to a fifth example embodiment of the present disclosure;

FIG. 34 is another perspective view of the spinal rod of FIG. 33, facing an opposite end of the spinal rod shown in FIG. 33;

FIG. 35 is a longitudinal top view of the spinal rod shown in FIG. 33;

FIG. 36 is a longitudinal bottom view of the spinal rod shown in FIG. 33;

FIG. 37 is a longitudinal side view of a first side of the spinal rod of FIG. 33;

FIG. 38 is a longitudinal side view of a second side of the spinal rod opposite the first side shown in FIG. 37;

FIG. 39 is a front end view of the spinal rod shown in FIG. 33;

FIG. 40 is a rear end view of the spinal rod shown in FIG. 33;

FIG. 41 is perspective view of a pair of spinal rods implanted in a patient and modified into an intra- or post-operative configuration, according to an embodiment of the present disclosure;

FIG. 42 is a perspective view of the spinal rod of FIG. 1 bent into a first example bend configuration;

FIG. 43 is a perspective view of the spinal rod of FIG. 1 bent into a second example bend configuration;

FIG. 44 is a perspective view of the spinal rod of FIG. 1 bent into a third example bend configuration;

FIG. 45 is a partial view of a spinal rod loaded in a bending tool, according to an embodiment of the present disclosure;

FIG. 46 is a partial view of the spinal rod loaded in the bending tool of FIG. 45 and at least partially bent by the bending tool;

FIG. 47 is a perspective view of spinal rod implantation system that includes a pair of spinal rods with markings thereon, according to an embodiment of the present disclosure.

### DETAILED DESCRIPTION

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

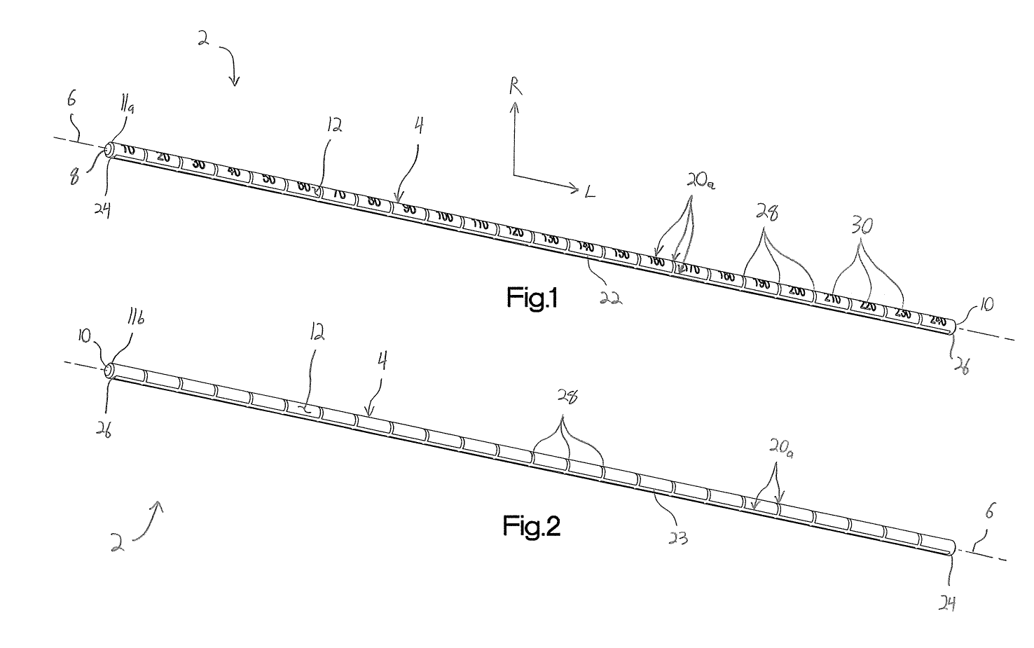

FIGS. 1 through 8 illustrate various view of a spinal rod 2 for implantation within a patient, according to a first example embodiment. The spinal rod 2 can include a body 4 that is elongate along a central rod axis 6 that extends along a longitudinal direction L that is orthogonal to a radial direction R. Thus, the central rod axis 6 can define a longitudinal axis of the body 4. The body 4 can also define a proximal end 8 and a distal end 10 spaced from the proximal end 8 in a distal direction along the central rod axis 6. Accordingly, the proximal end 8 is spaced from the distal end 10 in a proximal direction along the central rod axis 6. In the present disclosure, the proximal end 8 of the body 2 refers to the proximal-most end, or proximal terminus, of the body 4. Similarly, the distal end 10 of the body 2 refers to the distal-most end, or distal terminus, of the body 4. The body 4 can optionally define a first or proximal bevel 11a contiguous with the proximal end 8 and a second or distal bevel 11b contiguous with the distal end 10.

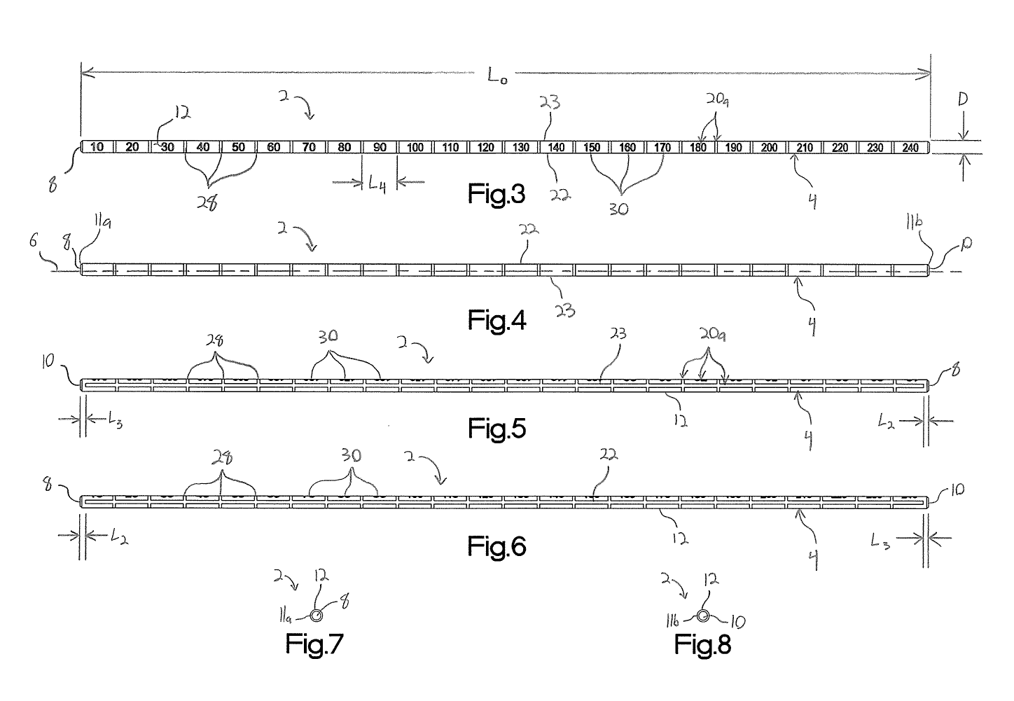

The body 4 can define an outer surface 12 that extends between the proximal and distal ends 8, 10. As shown, the outer surface 12 can extend from the proximal bevel 11a to the distal bevel 11b. The spinal rod 2 can have a diameter D between about 1.5 mm and about 7 mm (FIG. 3). The outer surface 12 can be cylindrical, although other configurations are within the scope of the present disclosure. The spinal rod 2 is depicted in a straight pre-operative configuration, although, in other embodiments, the spinal rods 2 can be at least partially curved or have an irregular shape in the pre-operative configuration. In one example embodiment, the pre-operative configuration, the spinal rod 2 can have a first or initial length L₀ between about 20 mm and about 900 mm (FIG. 3) measured from the proximal end 8 to the distal end 10 along the central rod axis 6. As used herein, the term “length” refers to a dimension measured along the longitudinal direction L. In the present embodiment, the initial length L₀ can be about 240 mm, by way of non-limiting example. It is to be appreciated that the foregoing rod 2 sizes are provided for illustrative purposes. Other rod 2 sizes, diameters D, and initial lengths L₀ are within the scope of the present disclosure, and can be selected based on the needs of the patient.

The spinal rod 2 is configured to be manipulated by a physician (or other technician) into an intra-operative or post-operative configuration during a surgical implantation procedure according to the various needs of the patient. For example, a physician can manipulate the spinal rod 2 into the intra-operative or post-operative configuration by bending the rod 2 to a desired shape. Accordingly, the body 4 can be formed of a material that is malleable so as to allow the spinal rod 2 to be bent to a predetermined curvature by a physician. The rod 2 can be bisected, severed, or otherwise trimmed (referred to herein as “cut”) to a desired second or final length L₁ before or after bending is completed. By way of non-limiting example, the body 4 of the spinal rod 2 can be formed of a biocompatible material, such as titanium, titanium alloys, stainless steel, cobalt chromium, polyetheretherketone (PEEK), nitinol, silicon nitride, or any combination of the foregoing materials.

The body 4 can include a pattern of reference markings 20a on the outer surface 12 thereof. The reference markings 20a can include a first longitudinal line 22 and a second longitudinal line 23 each elongate along the longitudinal direction L and extending between the proximal and distal ends 8, 10 of the body 4. Thus, the first and second longitudinal lines 22, 23 can be parallel with the central rod axis 6. The first and second longitudinal lines 22, 23 can be configured to provide a physician with visual indications of the straightness and/or curvature of the spinal rod 2, or various portions thereof, before, during, or after implantation. The second longitudinal line 23 can be spaced 180 degrees from the first longitudinal line 22 about the central rod axis 6. Thus, the first and second longitudinal lines 22, 23 can be oriented relative to one another such that, at each longitudinal location of the body 4, a straight line that is perpendicular to the central rod axis 6 can intersect the first and second longitudinal lines 22, 23 and the central rod axis 6. When the spinal rod 2 is straight, the two longitudinal lines 22, 23 can substantially define a plane that extends along the two longitudinal lines 22, 23. Thus, the physician can reference the first and second longitudinal lines 22, 23 to visualize the plane and associate the plane with the sagittal or coronal plane when manipulating the spinal rod 2 into the intra- or post-operative configuration.

Each of the longitudinal lines 22, 23 can define a line proximal end 24 and a line distal end 26 spaced from the line proximal end 24 in the distal direction along the central rod axis 6. As shown, the line proximal ends 24 can be offset from the proximal end 8 of the body 4 by a first offset distance L₂ along the longitudinal direction L, and the line distal ends 26 can be offset from the distal end 10 of the body 4 by a second offset distance L₃ along the longitudinal direction L. The first and second offset distances L₂, L₃ can be between about 1 mm and about 9 mm, although other offset distances are within the scope of the present disclosure. Additionally, in other embodiments, the proximal and distal ends 24, 26 of the longitudinal lines 22, 23 can be contiguous with the respective proximal and distal ends 8, 10 of the body 4 or at least with the proximal and distal bevels 11a, 11b.

The reference markings 20a can include hash marks 28 incrementally spaced from one another along the outer surface 12 at equidistant length intervals L₄ (referred to below as “intervals”) along the longitudinal direction L. In this manner, the hash marks 28 can provide a visual indication, or at least an approximation, of the length of the spinal rod 2 at each hash mark 28, as measured from the proximal end 8 of the body 4. The hash marks 28 can each comprise a transverse line oriented perpendicular to the central rod axis 6. Stated differently, each hash mark 28 can be elongate so as to extend circumferentially along the outer surface 12 about the central rod axis 6. The hash marks 28 can optionally extend around an entire circumference of the body 4. Each of the hash marks 28 can intersect the longitudinal lines 22, 23, although, in other embodiments, some or all of the hash marks 28 need not intersect the longitudinal lines 22, 23. Moreover, as each hash mark 28 has at least a marginal thickness along the longitudinal direction L, each interval L₄ can be defined between the longitudinal midpoints of each of the associated hash marks 28.

The intervals L₄ can be equivalent to standard units of length, such as inches, centimeters, millimeters, any combination thereof. In the first example embodiment, the hash marks 28 can be spaced at intervals L₄ of 10 mm (or 1 cm) along the longitudinal direction L. It is to be appreciated, however, that the intervals L₄ can be equivalent to any unit of length, including integer length units, fractional length units, or a combination of integer and fractional length units.

The reference markings 20a can include reference numbers 30 associated with at least some of the hash marks 28. In the present embodiment, each of the hash marks 28 is associated with a respective unique reference number 30. The numbers 30 can be oriented so as to read along the longitudinal direction L (i.e., the reference numbers 30 can be aligned along a single line that is parallel with the central rod axis 6); however, other orientations are possible. The numbers 30 can substantially denote or otherwise identify, or at least approximate, the length of the spinal rod 2 at the associated hash marks 28. In the present embodiment, a first 28a one of the hash marks 28 can be spaced from the proximal end 8 of the body 4 in the distal direction by the interval L₄. Also in the present embodiment, a final 28b one of the hash marks 28 can be spaced from the distal end 10 of the body 4 in the proximal direction by the interval L₄. Thus, a final 30b one of the reference numbers 30 may not necessarily be directly associated with a hash mark 28, but can identify, or at least approximate, the initial length L₀ of the spinal rod 2. Accordingly, in the present embodiment, each reference number 30 except the final reference number 30b can identify a distance along the longitudinal direction L from the proximal end 8 of the body 4 to the respective hash mark 28. Thus, the reference numbers 30 can be termed “callouts” or “length callouts” of the associated hash marks 28, or, in the case of the final number 30b, of the initial length L₀ of the spinal rod 2. It is to be appreciated that, in other embodiments, the final hash mark 28b can be located at or contiguous with the distal end 10 of the body 4.

With continued reference to FIGS. 1 through 8, each of the reference numbers 30 can be positioned on a proximal side of the associated hash mark 28 (i.e., spaced from the associated hash mark 28 in the proximal direction). In such embodiments, each number 30 can be centered at a longitudinal midpoint between the immediately adjacent hash marks 28. In this manner, the numbers 30 themselves can provide an indication of the longitudinal midpoints of each interval L₄ (i.e., the midpoint between adjacent hash marks 28). Furthermore, with the hash marks 28 and reference numbers 30 arranged in such a manner, the spinal rod 2 can be cut at one of the hash marks 28 while not disturbing or obscuring the associated number 30. Thus, the associated number 30 can indicate the final length L₁ of the spinal rod 2.

The reference markings 20 can be produced on the body 4 in various processes. In embodiments where the body 4 comprises titanium, the marking 20 can be produced in a titanium anodizing process. In other embodiments, the markings 20 can be produced in one or more processes that include etching, laser etching, chemical etching, photo etching, printing, inscribing, engraving, pad printing, stenciling, ink marking, epoxy ink marking, or any combination of the foregoing. In embodiments where the body 4 is curved in the pre-operative configuration, the body 4 can be bent into the curved pre-operative configuration prior to or after producing the markings 20 on the body 4.

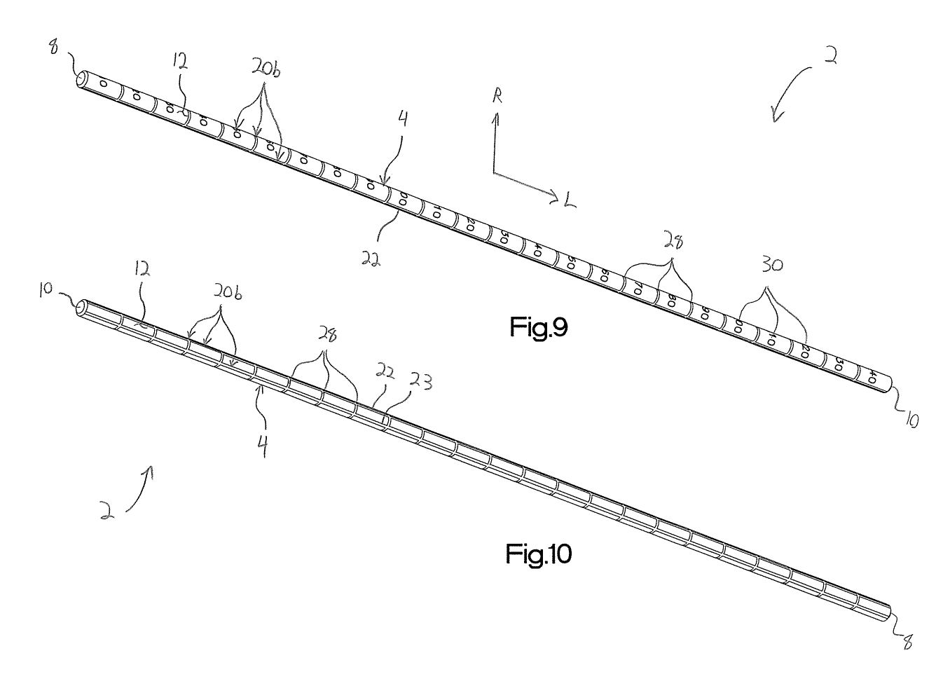

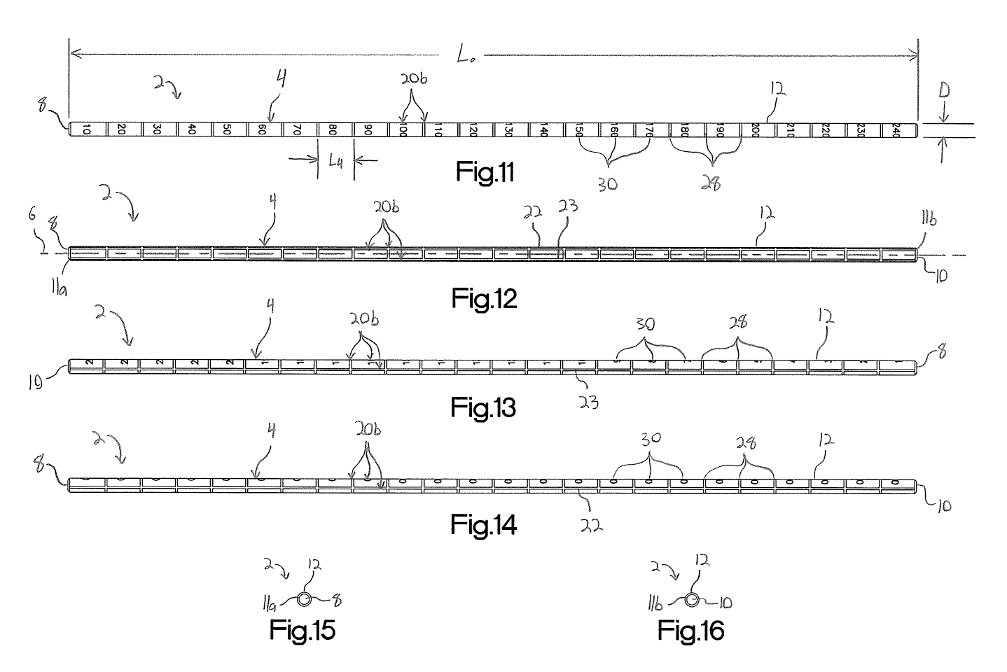

Referring now to FIGS. 9 through 16, various views of the spinal rod 2 are illustrated according to a second example embodiment. In particular, the spinal rod 2 of the second example embodiment employs a pattern of reference markings 20b that is different than the pattern of reference markings 20a of the first example embodiment, while the spinal rods 2 of the first and second example embodiments can otherwise be configured similarly to one another. Accordingly, the reference numbers 30 used in reference to FIGS. 1 through 8 can be duplicated in reference to FIGS. 9 through 16. Differences between the first and second example embodiments will now be set forth.

In the second example embodiment, the first and second longitudinal lines 22, 23 can be spaced apart from one another by about 90 degrees about the central rod axis 6. Thus, when the body 4 is straight, the physician can reference the first longitudinal line 22 to visual a first plane that extends therealong and along the central rod axis 6. The physician can also reference the second longitudinal line 23 to visualize a second plane that extends along both the second longitudinal line 23 and the central rod axis 6. The first plane can be associated with one of the sagittal and coronal planes and the second plane can be associated with the other of the sagittal and coronal planes while the physician manipulates the spinal rod 2 into the intra- or post-operative configuration. Also, as shown, in the second example embodiment, the first and second longitudinal lines can each extend from the first bevel 11a to the second bevel 11b. Stated differently, the proximal ends 24 of the longitudinal lines 22, 23 can be contiguous with the first bevel 11a, and the distal ends 26 of the longitudinal lines 22, 23 can be contiguous with the second bevel 11b. In this embodiment, the reference numbers 30 can be oriented to read in a circumferential direction (i.e., perpendicular to the longitudinal direction L).

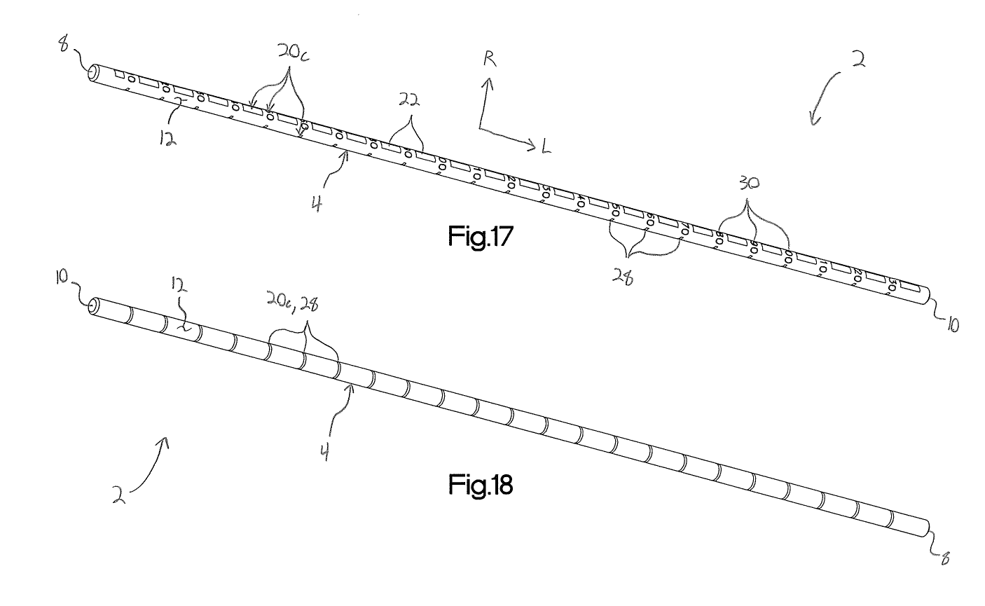

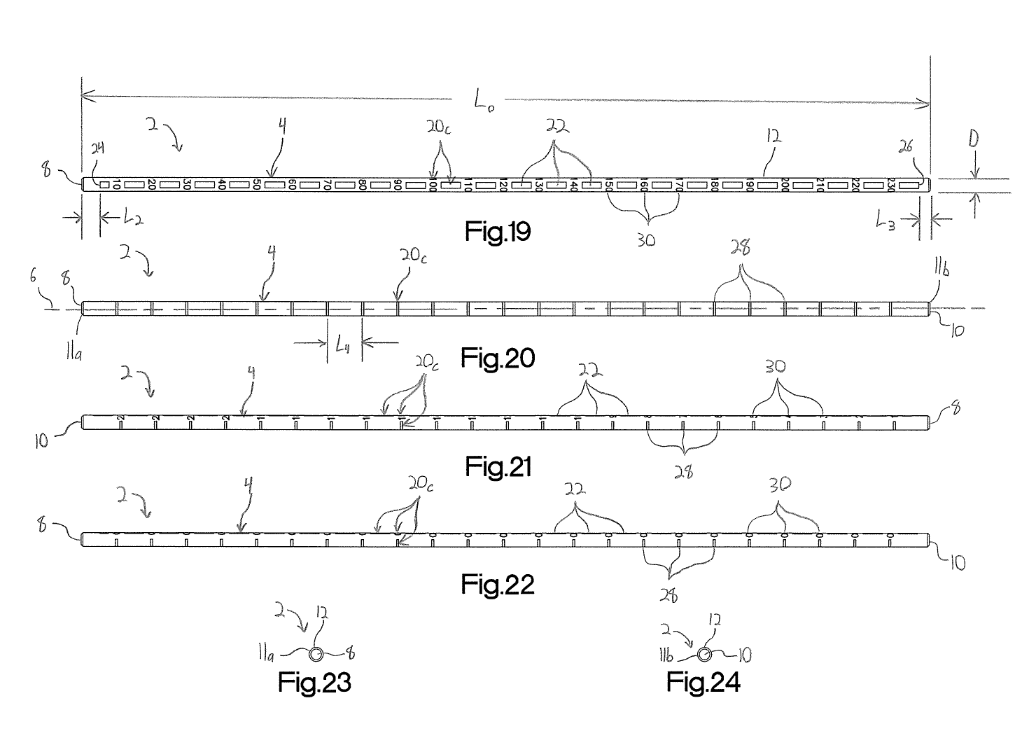

Referring now to FIGS. 17 through 24, various views of the spinal rod 2 are illustrated according to a third example embodiment that includes a pattern of reference markings 20c that is different than the patterns of the first and second example embodiments. The spinal rods 2 of the first, second and third example embodiments can otherwise be similar; thus, reference numbers of the first and second example embodiments can be duplicated with reference to FIGS. 17 through 24. Differences of the third example embodiment will now be set forth.

In the third example embodiment, the reference markings 20c include a single longitudinal line 22 that is dashed or broken, with the reference numbers 30 positioned between adjacent broken segments of the longitudinal line 22. In this embodiment, the reference numbers 30 are oriented to read in a circumferential direction, and the hash marks 22 extend circumferentially around the outer surface of the body 4 from one side of the associated reference numbers 30 to the other. The reference numbers 30 can be longitudinally aligned with the longitudinal midpoints of the associated hash marks 28. As with the first example embodiment, the proximal end 24 of the longitudinal line 22 can be offset from the proximal end 8 of the body 4 by a first offset distance L₂ along the longitudinal direction L, and the distal end 26 of the longitudinal line 22 can be offset from the distal end 10 of the body 4 by a second offset distance L₃ along the longitudinal direction L.

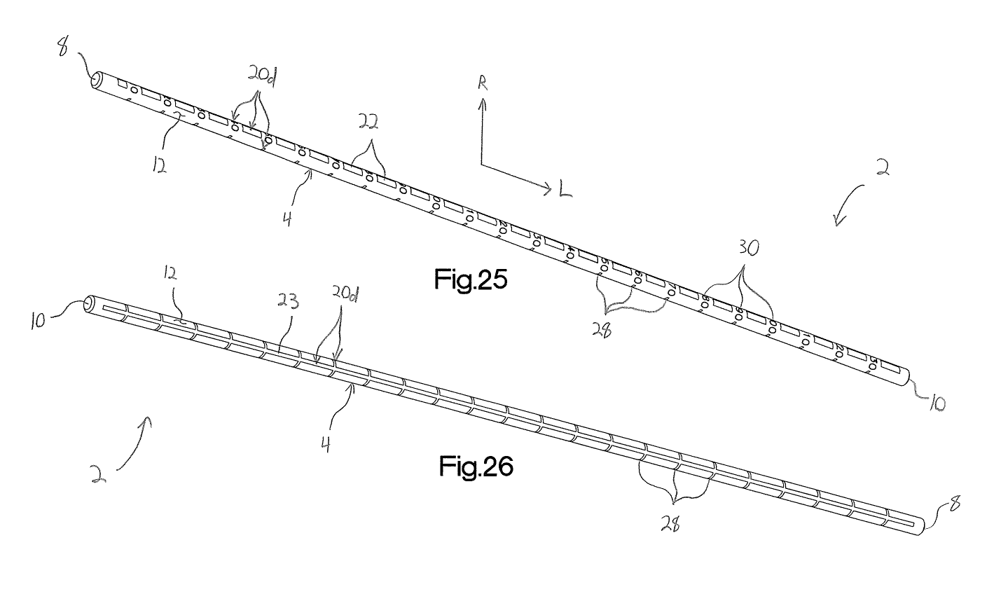

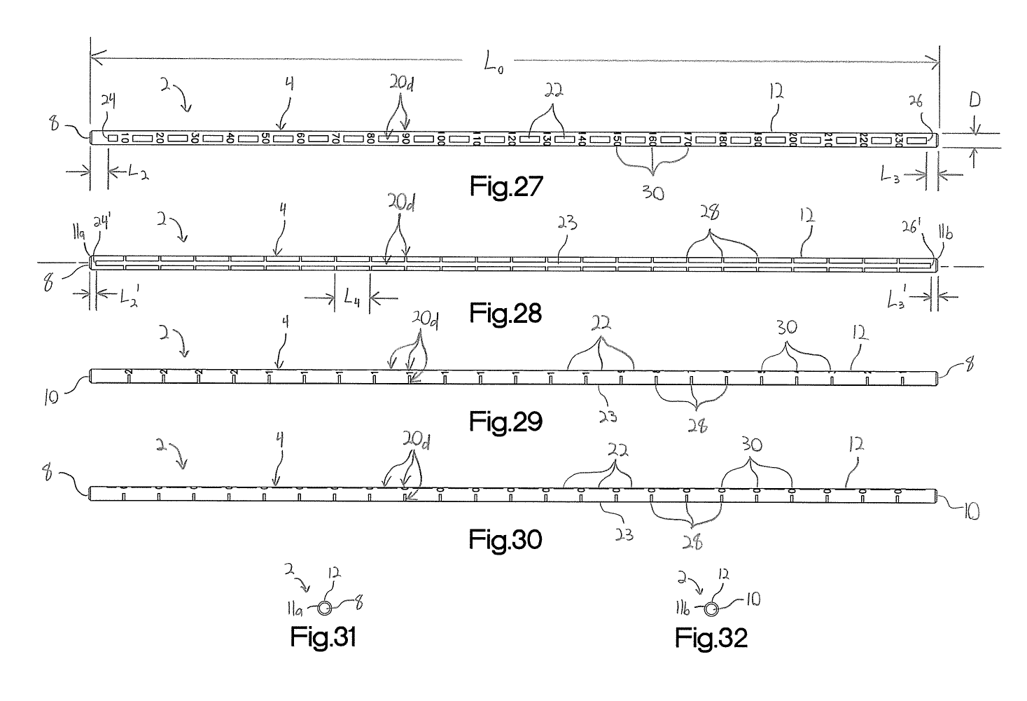

Referring now to FIGS. 25 through 32, various views of the spinal rod 2 are illustrated according to a fourth example embodiment that includes a pattern of reference markings 20d that is different than the patterns of the first, second, and third example embodiments. The spinal rods 2 of the first through fourth example embodiments can otherwise be similar; thus, reference numbers of the first through third example embodiments can be duplicated with reference to FIGS. 25 through 32.

The fourth example embodiment can be similar to the third example embodiment, with a primary difference being that the reference markings 20d of the fourth example embodiment can include a second longitudinal line 23 that extends in an unbroken manner between the proximal and distal ends 8, 10 of the body 4. A proximal end 24′ of the second longitudinal line 23 can be offset from the proximal end 8 of the body 4 by a third offset distance L₂′ that is different than the first offset distance L₂, and a distal end 26′ of the second longitudinal line 23 can be offset from the distal end 10 of the body 4 by a fourth offset distance L₃′ that is different than the second offset distance L₃. As with the first example embodiment, the first and second longitudinal lines 22, 23 of the present embodiment can be spaced 180 degrees apart from one another about the central rod axis 6.

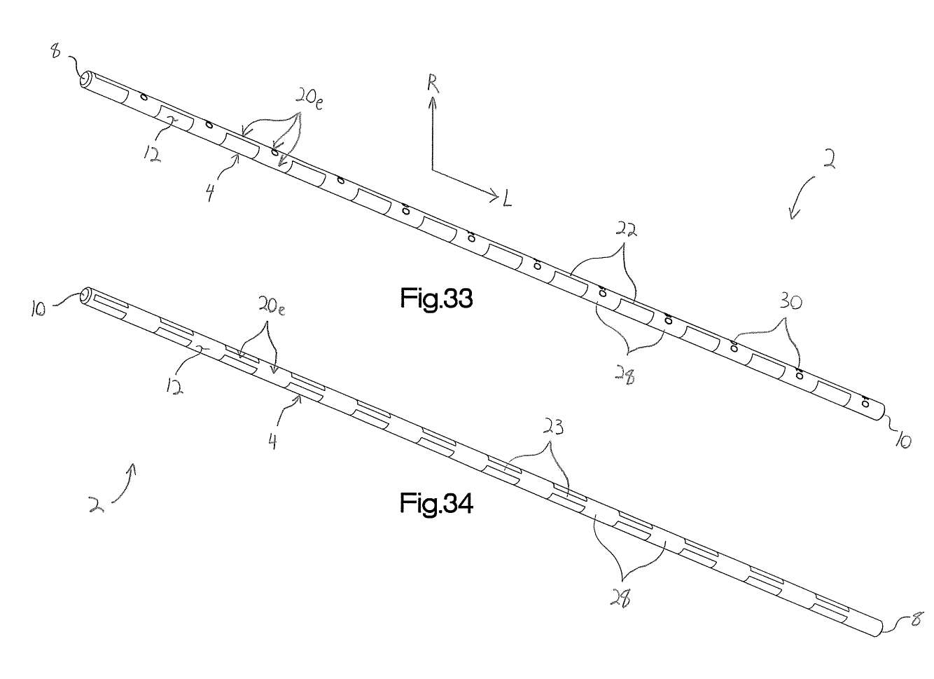

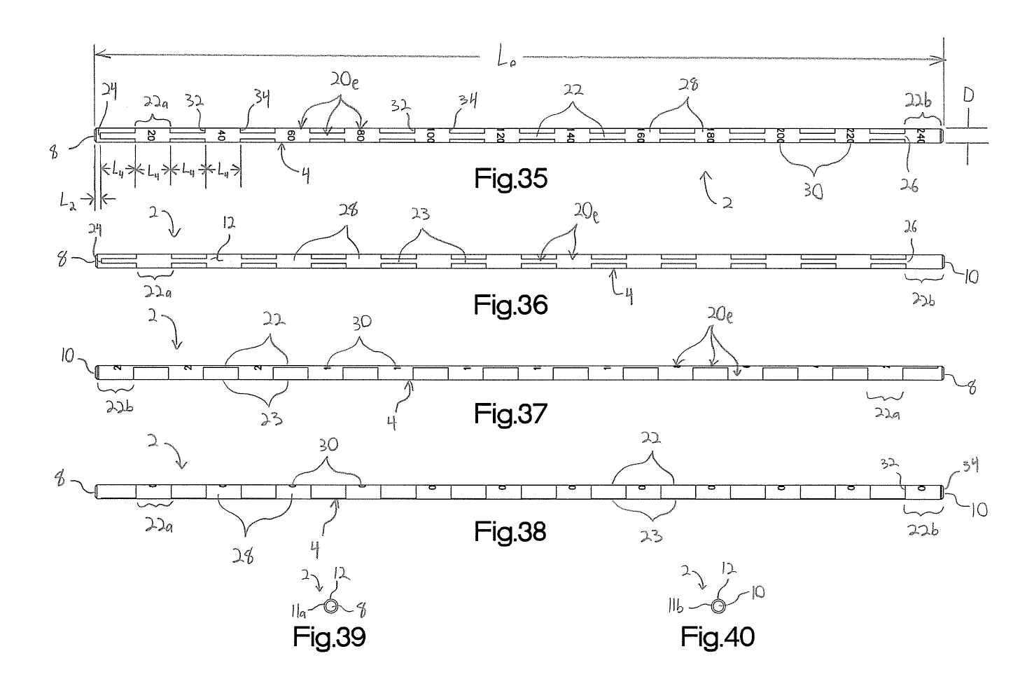

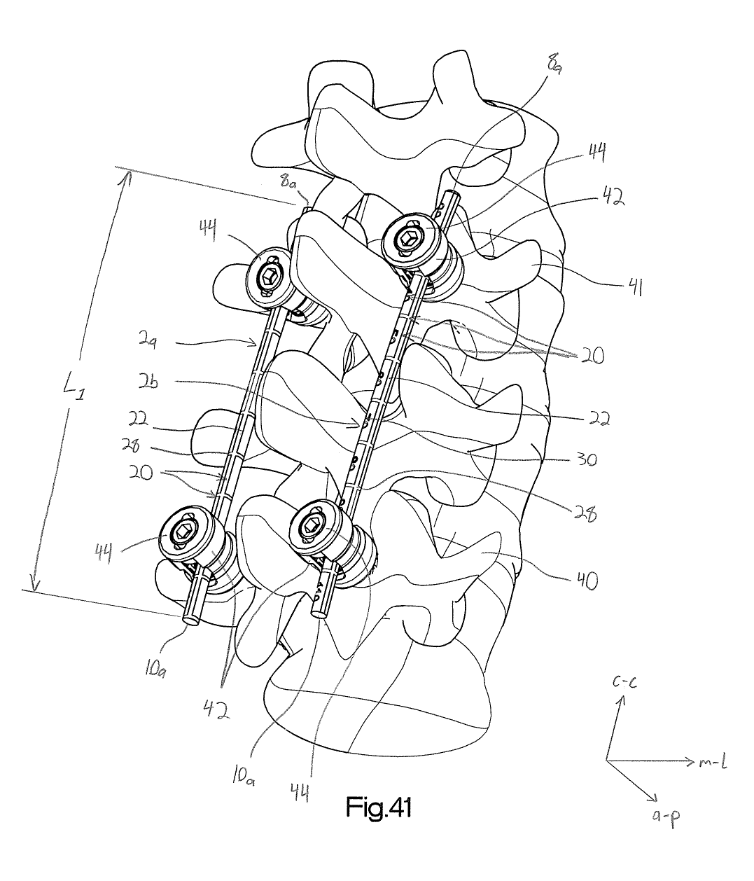

Referring now to FIGS. 33 through 40, various views of the spinal rod 2 are illustrated according to a fifth example embodiment that includes a pattern of reference markings 20e that is different than the patterns of the first through fourth example embodiments. The spinal rods 2 of the first through fifth example embodiments can otherwise be similar; thus, reference numbers of the first through fourth example embodiments can be duplicated with reference to FIGS. 33 through 40.

In the fifth example embodiment, the reference markings 20e can include first and second longitudinal lines 22, 23 spaced 180 degrees apart from one another about the central rod axis 6. The proximal ends 24 of each of the first and second longitudinal lines can be offset from the proximal end 8 of the body by a first offset distance L₂. In the present embodiment, the hash marks 28 can be elongated along the longitudinal direction L between a hash mark proximal end 32 and a hash mark distal end 34 spaced from the hash mark proximal end 32 in the distal direction. In such embodiments, each hash mark 28 may have a length equivalent to the interval L₄. Additionally, each adjacent pair of hash marks 28 can be spaced apart from one another along the longitudinal direction L by the interval L₄. Thus, the proximal end 32 of each hash mark (except, of course, the first hash mark 28a) can be spaced from the distal end 34 of the preceding hash mark 28 by the interval L₄. The proximal end 32 of the first hash mark 28a can be spaced from the proximal ends 24 of the first and second longitudinal lines along the longitudinal direction L by the interval L₄. The distal end 34 of the final hash mark 28b can substantially coincide with the distal end 10 of the body 4 or at least be contiguous with the distal bevel 11b. In the present embodiment, each hash mark 28, as well as the space between adjacent hash marks 28, represents the interval L₄.

The reference numbers 30 may be longitudinally positioned within the hash marks 28. Additionally, the reference numbers 30 may be circumferentially aligned with the first longitudinal line 22, may be oriented to read in a circumferential direction, and may be longitudinally centered at the longitudinal midpoint of the associated hash mark 28. Each of the reference numbers 30 in the present embodiment can denote, identify, or at least approximate the length of the spinal rod 2 measured from the proximal ends 24 of the longitudinal lines 22, 23 to the distal end 34 at the associated hash mark 28.

It is to be appreciated that the markings 20 of the spinal rod 2 are not limited to the patterns set forth above with reference to the foregoing example embodiments. The hash marks 28 and reference numbers 30 can be organized in any manner that provides a visual indication of the length of the spinal rod 2 at various longitudinal locations of the body 4. For example, the hash marks 28 can include major and minor hash marks, and the hash marks 28 can be spaced at irregular intervals along the longitudinal direction. Furthermore, the markings 20 can include three (3), four (4), or more than four longitudinal lines parallel with the central rod axis 6. The longitudinal lines can be organized in any manner that provides a visual indication of the straightness or curvature of the body 4.

Referring now to FIG. 41, a pair of spinal rods 2, including a first spinal rod 2a and a second spinal rod 2b, can be implanted in a patient to treat any of various spinal disorders. One or both of the first and second spinal rods 2a, 2b can be configured as disclosed above. In an example implantation procedure, screws or other anchors can be driven into pedicles of vertebrae that are to be adjusted or immobilized by the spinal rods 2a, 2b. When the anchors include screws driven into the pedicles, the screws are commonly referred to as “pedicle screws”. As shown, the vertebrae can include a first vertebra 40 and a second vertebra 41 spaced from one another along a cranial-caudal direction (c-c) that is perpendicular to a medial-lateral direction (m-l) and an anterior posterior direction (a-p). The pedicles screws on the same lateral side of the first and second vertebrae 40, 41 can be characterized as a “row” of screws. The screws, which are not visible in FIG. 41, can include features for connecting to the spinal rods 2a, 2b, such as anchor heads 42 with U-shaped channels in which the spinal rods 2a, 2b can be inserted and subsequently clamped by a fastener, such as a threaded nut, set screw, or locking cap 44. In this manner, each of the spinal rods 2a, 2b can be fixed to a row of screws to position or immobilize the vertebrae 40, 41 with respect to one another. Thus, the longitudinal directions L of the spinal rods 2a, 2b can each extend generally along the cranial-caudal direction. While FIG. 41 illustrates each row including only two (2) anchors with their associated anchor heads 42 and locking caps 44, it is to be appreciated that each row can include three (3) or more anchors affixed to a corresponding number of vertebrae.

The physician can manipulate one or both of the spinal rods 2a, 2b into an intra- or post-operative configuration prior to clamping the rods 2a, 2b to the anchor heads 42 with the locking caps 44. For example, the physician can bend the first spinal rod 2a into a first shape defining a first curvature, and can optionally bend the second spinal rod 2b into a second shape defining a second curvature. The first and second shapes can be determined based on the needs of the patient. In this manner, the spinal rod 2a, 2b can be shaped to maintain the vertebrae 40 in a desired orientation so as to correct the spinal disorder at hand, such as straightening a spine having an abnormal curvature, such as scoliosis, lordosis, or kyphosis. Accordingly, the spinal rods 2a, 2b can be bent in the sagittal plane to correct lordosis or kyphosis, and can be bent in the sagittal and coronal planes to correct scoliosis or other abnormal curvatures as needed. Additionally or alternatively, the spinal rods 2a, 2b may be positioned with respect to the screws to compress or distract adjacent vertebrae.

The shapes and curvatures of the spinal rods 2a, 2b can be at least partially determined based on scanned image data obtained of the patient's spine. Furthermore, in some procedures, the shapes and curvatures can be determined based on the positions of the anchor heads 42 affixed to the vertebrae during the procedure, or based on other factors discovered during the procedure. Once affixed to the anchor heads 42, the spinal rods 2a, 2b can be cut to the second or final length L₁ to minimize the amount of material implanted in the patient. The rods 2a, 2b can be cut at one or two locations outside the associated row. After the rods are cut, the remaining hash marks 28 and associated reference numbers 30 on the rods 2a, 2b can provide a visual indication of the final length L₁ of each rod 2, measured from a proximal cut end 8a to a distal cut end 10a of each rod 2a, 2b along their respective longitudinal directions L. To increase the accuracy of the visual indication, the physician can elect to cut the rods 2a, 2b at the hash marks 28. It is to be appreciated that the interval L₄ between the hash marks can optionally be sized such that any cut will be at least immediately adjacent a hash mark 28.

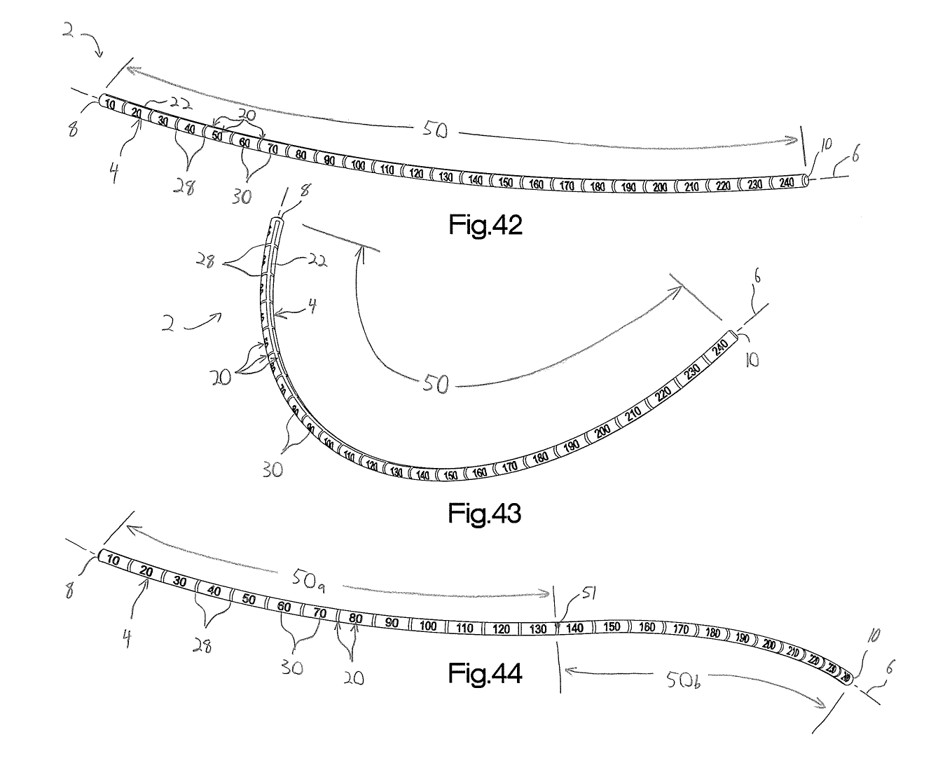

The reference markings 20 on the first and second spinal rods 2a, 2b can assist the physician with bending the spinal rods 2a, 2b into their respective desired shapes. As described above, the spinal rods 2a, 2b are malleable so as to be bent into the desired shapes for implantation, yet rigid enough to maintain the desired positions between the vertebrae. For example, as shown in FIGS. 42 through 44, a spinal rod 2, configured as set forth above (in this particular example, having a single longitudinal line 22), can be bent into various shapes to accommodate the needs of the patient. The spinal rod 2 can be manipulated so as to form one or more regions 50 defining respective bent shapes. As shown in FIGS. 42 and 43, the rod 2 can be manipulated so as to generally define a single region 50 that extends from the proximal end 8 to the distal end 10 of the rod 2 and has a bent shape. As shown in FIG. 44, the rod 2 can be manipulated so as to generally define a first region 50a defining a first bent shape and a second region 50b defining a second bent shape that is different than the first bent shape. The first region 50a can extend from the proximal end 8 of the rod 2 to an intermediate location 51 on the rod 2 in the distal direction, and the second region can extend from the intermediate location 51 to the distal end 10 of the rod 2 in the distal direction. However, in other embodiments, the rod 2 can be manipulated so as to define bent and straight regions, as desired.

With reference to FIG. 42, the spinal rod 2 can be bent so that the central rod axis 6 extends substantially entirely in only two (2) spatial dimensions (i.e., 2-dimensional or “2D” space) that define the “bending plane” of the spinal rod 2. The rod 2 can be bent so as to substantially define, or at least approximate, a single radius of curvature along its length or, as shown in FIG. 44, multiple different curvatures along the length of the rod 2. Furthermore, with reference to FIGS. 43 and 44, the spinal rod 2 can be bent so that the central rod axis 6 extends through three (3) spatial dimensions (i.e., 3-dimensional or “3D” space). In such embodiments, the spinal rod 2 can define multiple bending planes. In embodiments where it is desired to bend the spinal rod 2 in a single bending plane, the physician can optionally orient the longitudinal line 22 such that the longitudinal line 22 and the central rod axis 6 both extend along the bending plane. In this manner, the physician can visually reference the longitudinal line 22 “straight-on” (i.e., the longitudinal line 22 is centrally positioned on the outer surface 12 of the rod body 4) to ensure that the bending only occurs in a single bending plane; in particular, as long as the longitudinal line 22 remains substantially straight when viewed “straight-on”, the physician can confirm that the bending is occurring substantially only in the bending plane, which can be associated with the sagittal or coronal plane.

However, the patient's spine may require the spinal rod 2 to be bent in more than one bending plane. For example, the physician may require that the rod 2 be bent in both the sagittal and coronal planes when the rod 2 is in the intra- or post-operative configuration. FIG. 43 illustrates the spinal rod 2 bend into a shape defining a generally parabolic curvature that is not limited to a single bending plane (i.e., the central rod axis 6 extends through 3-dimensional space). FIG. 44 illustrates the spinal rod 2 bent into a shape defining a generally sinusoidal curvature that is also not limited to a single bending plane. In embodiments where the spinal rod 2 is bent in at least two (2) bending planes, the reference markings 20, including the longitudinal line 22, the hash marks 28, and the reference numbers 30 themselves, can provide a visual indication of the degree of curvature with respect to the bending planes. The hash marks 28 and associated reference numbers 30 can also provide an indication of the longitudinal locations at which the rod 2 is bent into one or more of the various shapes.

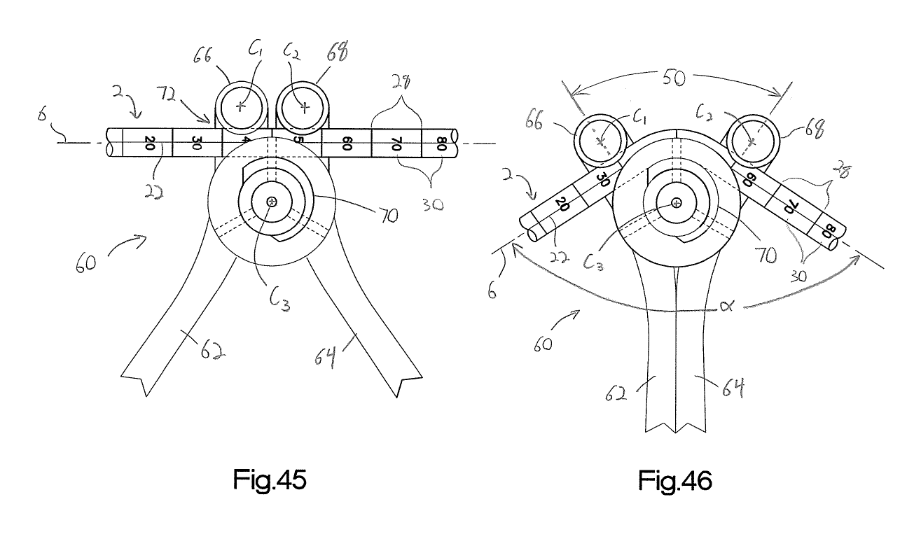

FIGS. 45 and 46 illustrate one possible tool for bending the spinal rod 2 into the intra- or post-operative configuration. As shown, the tool can be a hand-held rod-bending device 60, known in the art as “French rod bender” or simply a “French bender”; however, the markings 20 of the spinal rods 2 disclosed herein can provide similar benefits in other types of rod bending tools. The rod bender 60 is shown in FIG. 45 in an initial position and shown in FIG. 46 is a final position. The spinal rod 2 can be configured according to any of the embodiments set forth herein, and can include at least one longitudinal line 22, a plurality of hash marks 28, and reference numbers 30 associated with the hash marks 28. The rod bender 60 can include first and second handle arms 62, 64 pivotably coupled together in a manner such that squeezing the proximal ends of the handle arms 62, 64 together causes fulcrums 66, 68 at the respective distal ends of the arms 62, 64 to pivot away from one another along a single plane that defines the bending plane of the rod bender 60. The rod bender 60 can include a bending knob 70 optionally positioned over the pivot location of the handle arms 62, 64. Center points C₁, C₂ of the fulcrums 66, 68 can form the base of an isosceles triangle with the centerpoint C₃ of the bending knob 70 at the vertex. The centerpoint C₃ of the bending knob 70 defines the bending centerpoint of the rod bender 60, although other configurations are possible. The rod bender 60 can define a rod-receiving gap 72 located between the bending knob 70 and the fulcrums 66, 68 and configured to receive the spinal rod 2 therein. The physician can place the spinal rod 2 in the rod-receiving gap 72 while the rod bender 60 is in the initial position. With the spinal rod 2 in the rod-receiving gap 72, the physician can press the proximal portions of the handle arms 62, 64 together, which causes the fulcrums 66, 68 to pivot away from one other and against the spinal rod 2, bending the spinal rod 2 around the bending knob 70. For precision, many rod bending devices, such as the rod bender 60 depicted, are configured to bend the rod 2 in a single bending plane as the rod bender 60 moves from the initial to the final position, which bending plane can be defined by the directions in which the handle arms 62, 64 and the fulcrums 66, 68 pivot. The rod bender 60 can essentially define a second plane that is orthogonal to the bending plane and parallel with a straight line extending through the centerpoints C₁, C₂ of the fulcrums 66, 68. The bending plane and the second plane can be associated with the sagittal and coronal planes.

With such bending tools, the characteristics of each individual bend performed therewith are limited. For example, with respect to the rod bender 60 shown, each individual bending operation bends the spinal rod 2 substantially only in the bending plane. Additionally, as shown in FIG. 46, the region 50 of the rod 2 in which the rod 2 is bent, as well as the maximum of the bend angle α imparted by the tool, can be limited by such parameters as the extent of pivoting motion of the fulcrums 66, 68, the shape and/or radius of the bending knob 70, and the distance between the fulcrums and the centerpoint C₃ of the bending knob 70, for example. Thus, to achieve the desired final bent shape of the spinal rod 2 in the intra- or post-operative configuration, the physician may need to perform a plurality of bending operations to work or manipulate the spinal rod 2 into the desired shape. For example, to bend the rod 2 so as to substantially define, or at least approximate, a single wide radius of curvature along its length, as shown in FIG. 42, the physician can perform a plurality of wide radius bends in succession along the length of the spinal rod 2. Moreover, to form a more profound bent shape (i.e., a bend having a narrowed radius of curvature), as shown in FIG. 43, the physical can perform a plurality of relatively sharp radius bends in succession along the length of the spinal rod 2. Furthermore, to bend the rod 2 in two (2) or more bending planes, as shown in FIGS. 43 and 44, the physician can form a first region having a first bent shape via one or more bending actions with the rod bender 60, then translate the spinal rod 2 relative to the rod bender 60, rotate the spinal rod 2 about the central rod axis 6 relative to the rod bender 60 by a non-180 degree angle, and forming a second region having a second bent shape via one or more additional bending actions with the rod bender 60. In this manner, the physician can use the rod bender 60 to manipulate the spinal rod 2 into a variety of 2-dimensional or 3-dimensional shapes.

The reference markings 20 disclosed herein can assist the physician with shaping the spinal rod 2 into the desired intra- or post-operative configuration in a number of ways. For example, with reference to the rod bender 60 shown in FIGS. 45 and 46, the hash marks 28 and associated reference numbers 30 can allow the physician to visually identify the length of the spinal rod 2 at which the bending centerpoint is located. Thus, if the physician has predetermined the longitudinal positions of one or more bends to be imparted to the spinal rod 2, the hash marks 28 and reference numbers 30 can provide the physician with visual references on the spinal rod 2 for alignment with the bending centerpoint defined by the tool 60. For example, if the physician determines that the rod 2 requires a bend centered at 40 mm from the proximal end 8 of the rod 2 along the longitudinal direction L, the physician can position the rod 2 in the rod-receiving gap 72 such that the hash mark 28 associated with the 40 mm callout reference number 30 is aligned with the centerpoint C₃ of the bending knob 70 (or a midpoint between the fulcrums 66, 68). Thus, the reference markings 20 allow the physician to more accurately position the spinal rod 2 in the rod bender 60 to accomplish the predetermined bends.

Additionally, the longitudinal line 22 can provide the physician with a visual indication of the bend angle α in the bending plane. To maximize this indication, the physician can orient the rod 2 about the central rod axis 6 at a first orientation, wherein the longitudinal line 22 is centered within the rod-receiving gap 72 (i.e., such that a straight line that is perpendicular to, and intersects, both of the central rod axis 6 and the longitudinal line 22 is also orthogonal to the bending plane of the rod bender 60). Furthermore, as set forth above, the physician can also orient the rod 2 within the rod bender 60 at a second orientation, wherein the longitudinal line 22 and the central rod axis 6 are both coextensive with the bending plane (i.e., 90 degrees offset from the first orientation). In the second orientation, as the physician works the rod 2 into the intra- or post-operative configuration, the physician can visually reference the longitudinal line 22 “straight-on” to identify the straightness or curvature of the rod 2 in the second plane. In embodiments where the rod markings 20 include a first longitudinal line 22 and a second longitudinal line 23 spaced 90 degrees apart from one another about the central rod axis 6 (such as shown in FIGS. 9 through 16), the physician can orient the rod 2 in the rod bender 60 such that the first longitudinal line 22 is in the first orientation and the second longitudinal line 23 is in the second orientation. Thus, while the physician performs bends with the rod bender 60, the first and second longitudinal lines 22, 23 can provide a visual indication of the curvature of the rod 2 in relation to the sagittal and coronal planes.

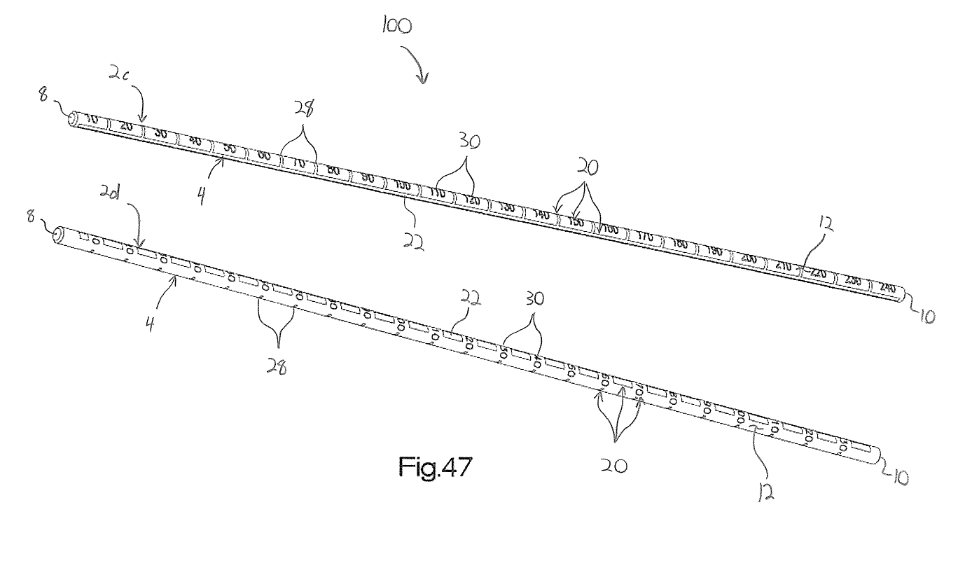

Referring now to FIG. 47, a spinal rod implantation system 100 can include a first or template rod 2c and a second or implantation rod 2d. The template and implantation rods 2c, 2d can each be configured according to any of the embodiments disclosed herein, and can each include reference markings 20 that include one or more longitudinal lines 22, 23, hash marks 28, and reference numbers 30 as set forth above. The pattern of reference markings 20 on the template and implantation rods 2c, 2d are shown as being different; however, in other embodiments, the template and implantation rods 2c, 2d can have the same markings 20.

The template rod 2c can be more malleable than the implantation rod 2d, which can allow the template rod 2c to be manually bent by the physician without the use of bending tools. By way of non-limiting example, the template rod 2c can be formed of aluminum or an aluminum alloy, and the implantation rod 2d can be formed of titanium or a titanium alloy. Thus, the implantation rod 2d can be more rigid so as to maintain the vertebrae in the desired orientation, or even to influence a predetermined repositioning of the vertebrae with respect to each other, once the implantation rod 2d is affixed to the respective anchor heads.

During an implantation procedure, the physician can prepare the implantation rod 2d for implanting within a patient in the following manner. Once the bone anchors are inserted into the vertebrae, the physician can place the template rod 2c next to the anchor heads of the row in which the implantation rod 2d is to be implanted, and then manually bend or deform the template rod 2c so that the template rod 2c can be coupled to the row of anchor heads in a manner to produce the desired positioning or repositioning of the vertebrae. This bending process can include bending the template rod 2c so as to form at least one region 50 defining a first bent shape, and can optionally include forming a plurality of regions 50 of the template rod 2c each having a bent shape. The physician can temporarily place the template rod 2c in an implantation position adjacent the spine of the patient. For example, the physician can temporarily seat the bent template rod 2c within the U-shaped channels of the anchor heads in the row and further bend or deform the template rod 2c as needed into a final template shape. Additionally, the physician can cut or sever the template rod 2c to the final length L₁. In other embodiments, however, the physician need not actually cut the template rod 2c to the final length L₁, but can instead denote or otherwise identify one or more cutting locations on the template rod 2c at which locations the template rod 2c could be cut to the final length L₁.

Once the template rod 2c is in the final template shape, the physician can observe the reference markings 20 on the template rod 2c to plan the bending of the implantation rod 2d in a bending tool, such as the rod bender 60 of FIGS. 45 and 46. For example, the physician can identify on the template rod 2c longitudinal locations of interest including, but not limited to, the cut locations and the longitudinal locations of the bent regions 50 and the midpoints thereof. The physician can also reference the hash marks 28 and associated reference numbers 30 on the template rod 2c adjacent such locations of interest to identify, determine, or otherwise approximate the respective lengths of the template rod 2c from the proximal end 8 of the rod 2 to the locations of interest along the longitudinal direction L. Additionally, the physician can observe the one or more longitudinal lines 22 on the bent template rod 2c to identify the bend angle α and/or radius of curvature of the bent regions, as well as to obtain an indication of the curvature of the template rod 2c in one or both of the sagittal and coronal planes.

The physician can compare the reference markings 20 of the template rod 2c in the final template shape with the reference markings 20 of the implantation rod 2d prior to and/or while bending the implantation rod 2d into the intra- or post-operative configuration. For example, by referencing the hash marks 28 and associated reference numbers 30 on the implantation rod 2d, the physician can identify locations on the implantation rod 2d that correspond to the locations of interest on the template rod 2c. On the implantation rod 2d, the lengths from the proximal end 8 of the rod 2d to each of the locations of interest can be substantially equivalent to those on the template rod 2c. Therefore, to form a bent region 50 on the implantation rod 2d that is similar to an associated bent region 50 of the template rod 2c, the physician can use the hash marks 28 and reference numbers 30 to position the implantation rod 2d in the rod bender 60 such that the bending centerpoint of the rod bender 60 is aligned with a location on the implantation rod 2d that corresponds to the longitudinal midpoint of the associated bent region 50 of the template rod 2c.

Additionally, the physician can orient the implantation rod 2d in the rod bender 60 so that the one or more longitudinal lines 22 can provide an indication of the bending curvature of the implantation rod 2d in one or more of the bending plane and the second plane during bending. Thus, while forming each bent region of the implantation rod 2d with the rod bender 60, the physician can compare the one or more longitudinal lines 22 of each of the template and implantation rods 2c, 2d until the curvature of the implantation rod 2d in the bending plane and/or the second plane is substantially equivalent to, or at least approximates, that of the template rod 2d in the final template shape. These processes can be repeated until the bent regions 50 of the implantation rod 2d define bent shapes that are substantially equivalent to, or at least to approximate, the bent shapes of the bent regions 50 of the template rod 2c. Additionally, by referencing the markings 20 on the template and implantation rods 2c, 2d, the physician can cut the implantation rod 2d at one or more longitudinal locations that correspond to those at which the template rod 2c was cut or denoted for cutting. In this manner, the physician can compare or otherwise reference the markings 20 on the template and implantation rods 2c, 2d while bending, deforming or otherwise shaping the implantation rod 2d so that the intra- or post-operative shape of the implantation rod 2d is substantially equivalent to, or at least approximates, the final template shape of the template rod 2c. It is to be appreciated that the shape of the implantation rod 2c can be said to be “substantially equivalent” to the final template shape of the template rod 2c when the implantation rod 2d is shaped sufficiently to be attached to the anchor heads of the associated row.

Although various embodiments have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, methods and steps described in the specification. As one of ordinary skill in the art will readily appreciate from the present disclosure, processes, machines, manufacture, composition of matter, methods, or steps, presently existing or later to be developed that perform substantially the same function or achieve substantially the same result as the corresponding embodiments described herein may be utilized.

It will be appreciated by those skilled in the art that various modifications and alterations to the embodiments described herein can be made without departing from the broad scope of the appended claims. Some of these have been discussed above and others will be apparent to those skilled in the art.

We claim:

1. A method of preparing a spinal rod, comprising:
obtaining a body elongate along a central rod axis, the body including a proximal end and a distal end spaced from each other along a longitudinal direction to as to define the spinal rod, the body defining an outer surface extending between the proximal and distal ends, wherein the body comprises a material that is malleable so as to allow the spinal rod to be bent to a predetermined curvature;
producing at least one longitudinal line on the outer surface, the at least one longitudinal line elongate along the longitudinal direction and parallel with the central rod axis;
producing a plurality of harsh marks incrementally spaced along the outer surface; and
producing reference numbers on the outer surface, each of the reference numbers identifying a distance along the longitudinal direction from the proximal end of the body to a respective one of the hash marks.

2. The method of claim 1, wherein the hash marks include:
a first hash mark located at the proximal end of the body;
a final hash mark located adjacent the distal end of the body; and
a series of intermediate hash marks located between the first and final hash marks.

3. The method of claim 1, wherein one or more of the at least one longitudinal line, the plurality of hash marks, and the reference numbers is created in a titanium anodizing process.

4. The method of claim 1, further comprising one or more of etching, laser etching, chemical etching, photo etching, printing, inscribing, engraving, pad printing, stenciling, ink marking, and epoxy ink marking one or more of the at least one longitudinal line, the plurality of hash marks and the reference numbers on the body.

5. The method of claim 1, wherein the at least one longitudinal line extends continuously between the proximal and distal ends of the body.

6. The method of claim 1, wherein the at least one longitudinal line includes a first longitudinal line and a second longitudinal line each elongated along the longitudinal direction and oriented parallel with the central rod axis.

7. The method of claim 6, further comprising orienting the first and second longitudinal lines relative to one another such that, at each longitudinal location of the body, a straight line that is perpendicular to the central rod axis intersects the first and second longitudinal lines and the central rod axis.

8. The method of claim 7, further comprising intersecting each of the hash marks with the first and second longitudinal lines.

9. The method of claim 7, further comprising intersecting at least a majority of the hash marks with the first and second longitudinal lines.

10. The method of claim 9, further comprising aligning the reference numbers along a single line that is parallel with the central rod axis; and
orienting the reference numbers such that each of the reference numbers reads in a direction perpendicular to the central rod axis.

11. The method of claim 10, further comprising locating each of the reference numbers on a proximal side of the respective one of the hash marks.

12. The method of claim 11, further comprising spacing the hash marks at equidistant length intervals along the body.

13. The method of claim 12, wherein a distal-most one of the hash marks is spaced from the distal end of the body by one of the equidistant length intervals.

14. The method of claim 1, wherein the obtaining step comprises obtaining the rod with the body being curved so as that the central rod axis is arcuate.

15. The method of claim 1, further comprising bending the body so that the central rod axis is arcuate prior to the producing steps.

16. A method of preparing a spinal rod for implantation, the method comprising:
obtaining a template rod and an implantation rod each defining a central axis and having an outer surface that defines:
at least one line elongated along a longitudinal direction that is parallel with the central axis;
hash marks spaced at intervals along the longitudinal direction; and
reference numbers identifying a distance from a proximal end of the associated rod to a respective one of the hash marks, measured along the longitudinal direction;
bending the template rod so as to form a first region defining a first bent shape;
after bending the template rod, comparing at least one of the hash marks and associated reference numbers of the template rod with at least one of the hash marks and associated reference numbers of the implantation rod so as to locate a second region of the implantation rod for bending;
bending the implantation rod at the second region so as to form a second bent shape; and
comparing a curvature of the at least one line of the template rod with a curvature of the at least one line of the implantation rod while bending the implantation rod until the second bent shape is substantially equivalent to the first bent shape.

17. The method of claim 16, further comprising, prior to the second bending step, placing the implantation rod in a bending tool and orienting the implantation rod in the bending tool such that the bending tool is configured to bend the implantation rod in a first plane that is perpendicular to a second plane coextensive with the central axis and the at least one line, wherein the second bending step comprising bending the implantation rod in the first plane with the bending tool so as to form the second bent region defining the second shape.

18. The method of claim 17, wherein the bending tool comprised a bending knob defining a bending centerpoint, and the placing step comprises aligning a longitudinal center of the second region of the implantation rod with the bending centerpoint of the tool.

19. The method of claim 16, further comprising,
after the first bending step, temporarily placing the template rod in an implantation position adjacent the spine of a patient;
denoting a first longitudinal location on the template rod;
referencing at least one of the hash marks and associated reference numbers adjacent the longitudinal location so as to determine a first length measured from the proximal end of the template rod to the longitudinal location along the longitudinal direction;
after the second bending step, referencing the hash marks and associated reference numbers of the implantation rod so as to identify on the implantation rod a second longitudinal location at which a second length, measured from the proximal end of the implantation rod to the second longitudinal location along the longitudinal direction, is substantially equivalent to the first length; and
cutting the implantation rod at the second longitudinal location.

20. The method of claim 19, wherein the denoting step comprises severing the template rod at the first longitudinal location.
